(19) United States
(12) Patent Application Publication
(10) Pub. No.: US 2016/0030006 A1
(43) Pub. Date: Feb. 4, 2016

(54) CHARACTERISTIC-VALUE CALCULATING DEVICE, CHARACTERISTIC-VALUE CALCULATING METHOD, AND RECORDING MEDIUM

(71) Applicant: OKI ELECTRIC INDUSTRY CO., LTD., Tokyo (JP)

(72) Inventors: Daisuke OKUYA, Tokyo (JP); Kurato MAENO, Tokyo (JP); Michiyo MATSUI, Tokyo (JP)

(73) Assignee: Oki Electric Industry Co., Ltd., Tokyo (JP)

(21) Appl. No.: 14/776,735

(22) PCT Filed: Oct. 1, 2013
(86) PCT No.: PCT/JP2013/076641

§ 371 Date: Sep. 14, 2015

(30) Foreign Application Priority Data
Apr. 26, 2013 (JP) 2013-093529

(51) Int. Cl. A61B 8/08; G01S 15/88; G01S 17/88

(52) U.S. Cl. CPC A61B 8/488; G01S 17/88; G01S 15/88; A61B 8/5223

(57) ABSTRACT

[Object] To provide a characteristic-value calculating device, a characteristic-value calculating method, and a recording medium that can extract a characteristic value for more accurately recognizing the state of a moving object, such as a human, from a Doppler signal.

[Solution] A characteristic-value calculating device includes an acquiring unit that acquires a Doppler signal, an extracting unit that extracts a time-series signal constituted of a predetermined frequency component from the Doppler signal acquired by the acquiring unit, a selecting unit that selects a signal value at a predetermined interval from the time-series signal extracted by the extracting unit, and a calculating unit that calculates higher-order local autocorrelation based on the signal value selected by the selecting unit.

### TECHNICAL FIELD

The present invention relates to a characteristic-value calculating device, a characteristic-value calculating method, and a recording medium.

### BACKGROUND ART

In recent years, there has been developed a technology in which a sensor is installed in a specific area, such as inside a building, for detecting, for example, the state of a person present at that location.

For example, Patent Literatures 1 and 2 below disclose technologies for detecting the presence of a person inside a building based on body movement or biological information obtained from a sensor. More specifically, Patent Literature 1 discloses a technology for estimating the state in the building from three states, namely, unmanned, rest, and activity states, by performing a threshold determination process based on the intensity of a signal of a Doppler sensor radiated into the room and a variance value. Patent Literature 2 discloses a technology for extracting a respiration component, a heartbeat component, and a body-movement component by performing frequency conversion and a filtering process on a signal from a pressure sensor attached to a life support device so as to detect the absence or presence from these components or to detect an emergency situation.

Furthermore, Patent Literature 3 below discloses a technology for differentiating sleeping states and performing problem detection intended for performing monitoring during sleeping. More specifically, Patent Literature 3 discloses a technology for detecting, for example, respiration, a roll-over, or a fall-off from a bed by radiating a signal from a Doppler sensor toward a person sleeping on the bed and then performing a threshold determination process based on information, such as an operating time, speed, and direction, obtained from the Doppler signal.

### CITATION LIST

### Patent Literature

Patent Literature 1: JP 2011-215031A

Patent Literature 2: JP 2004-174168A

Patent Literature 3: JP 2012-5745A

### SUMMARY OF INVENTION

### Technical Problem

However, the technologies disclosed in Patent Literatures 1 to 3 described above are problematic in that, since only a part of the human biological information, such as the signal intensity, the variance, the frequency component, and the speed relative to the sensor, is used, false detection may occur if there is disturbance that resembles these pieces of information within the area.

For example, in the technology disclosed in Patent Literature 1 described above, if there is a small animal, such as a pet or an insect, it is difficult to distinguish the small animal from a human solely based on the signal intensity and the variance thereof. In the technology disclosed in Patent Literature 2 described above, if there is an electrical household device that operates in a cycle similar to the human respiration cycle, it is not possible to detect accurate biological information. Furthermore, with regard to the technology disclosed in Patent Literature 3, since the technology is specialized for monitoring a person on a bed, human respiration or body movement can be extracted with respect to the limited space on the bed. However, in view of applying the technology to a wide range, such as the entire interior of the building, the technology is problematic in that differentiation from disturbance is not taken into consideration.

In view of the problems mentioned above, an object of the present invention is to provide a new and improved characteristic-value calculating device, characteristic-value calculating method, and recording medium that can extract, from a Doppler signal, a characteristic value for more accurately recognizing the state of a moving object, such as a human.

### Solution to Problem

To solve the problem, according to an aspect of the present invention, there is provided a characteristic-value calculating device including: an acquiring unit that acquires a Doppler signal; an extracting unit that extracts a time-series signal constituted of a predetermined frequency component from the Doppler signal acquired by the acquiring unit; a selecting unit that selects a signal value at a predetermined interval from the time-series signal extracted by the extracting unit; and a calculating unit that calculates higher-order local autocorrelation based on the signal value selected by the selecting unit.

The characteristic-value calculating device may further include: a vector normalization unit that performs vector normalization on the signal value selected by the selecting unit. The calculating unit may calculate the higher-order local autocorrelation based on the signal value normalized by the vector normalization unit.

The characteristic-value calculating device may further include: a dimensional compression unit that performs dimensional compression on the higher-order local autocorrelation calculated by the calculating unit.

The characteristic-value calculating device may further include: a preprocessing unit that performs predetermined signal processing on the Doppler signal acquired by the acquiring unit. The extracting unit may extract the time-series signal from the Doppler signal signal-processed by the preprocessing unit.

The preprocessing unit may perform offset adjustment on the Doppler signal acquired by the acquiring unit.

The extracting unit may extract the time-series signal constituted of a frequency component arising from human movement from the Doppler signal acquired by the acquiring unit.

The characteristic-value calculating device may further include: a recognizing unit that recognizes a state of a space subjected to observation of the Doppler signal acquired by the acquiring unit based on the higher-order local autocorrelation calculated by the calculating unit.

The recognizing unit may recognize at least one of unmanned, rest, and activity states as the state of the space.

The characteristic-value calculating device may further include: an observing unit that observes the Doppler signal and outputs the Doppler signal to the acquiring unit.

To solve the problem, according to another aspect of the present invention, there is provided a characteristic-value calculating method including: a step for acquiring a Doppler signal; a step for extracting a time-series signal constituted of a predetermined frequency component from the acquired Doppler signal; a step for selecting a signal value at a predetermined interval from the extracted time-series signal; and a step for calculating higher-order local autocorrelation based on the selected signal value.

To solve the problem, according to another aspect of the present invention, there is provided a recording medium having a program stored therein, the program causing a computer to execute: a step for acquiring a Doppler signal; a step for extracting a time-series signal constituted of a predetermined frequency component from the acquired Doppler signal; a step for selecting a signal value at a predetermined interval from the extracted time-series signal; and a step for calculating higher-order local autocorrelation based on the selected signal value.

### Advantageous Effects of Invention

As described above, according to the present invention, a characteristic value for more accurately recognizing the state of a moving object, such as a human, can be extracted from a Doppler signal.

### BRIEF DESCRIPTION OF DRAWINGS

FIG. 1 is a diagram schematically illustrating a state recognizing system according to an embodiment of the present invention.

FIG. 2 is a block diagram illustrating the configuration of the state recognizing system according to an embodiment of the present invention.

FIG. 3 is a flowchart illustrating an operation of the state recognizing system according to an embodiment of the present invention.

FIG. 4 is a diagram illustrating frequency distribution of average frequencies by FFT in a one-second interval.

FIG. 5 is a diagram illustrating frequency distribution of average frequencies by FFT in a ten-second interval.

FIG. 6 is a diagram illustrating frequency distribution of a first principal component of higher-order local autocorrelation in a one-second interval in accordance with an embodiment of the present invention.

### DESCRIPTION OF EMBODIMENTS

A preferred embodiment of the present invention will be described in detail below with reference to the appended drawings. It should be noted that, in the present description and the drawings, components having substantially identical functions and configurations will be given the same reference signs, and redundant descriptions thereof will be omitted.

### 1. Overview of State Recognizing System According to Embodiment of Present Invention

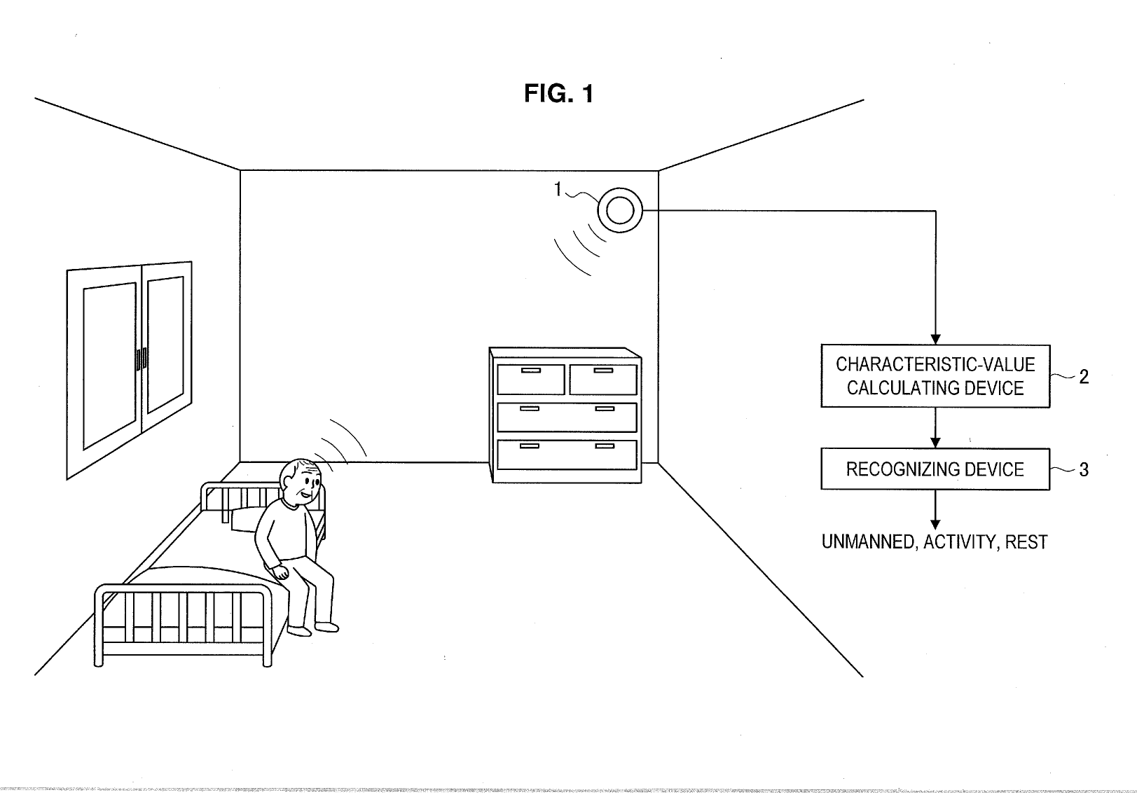

First, an overview of a state recognizing system according to an embodiment of the present invention will be described with reference to FIG. 1.

FIG. 1 is a diagram schematically illustrating the state recognizing system according to the embodiment of the present invention. As shown in FIG. 1, the state recognizing system according to the embodiment of the present invention has a Doppler sensor 1,a characteristic-value calculating device 2, and a recognizing device 3. As shown in FIG. 1, the Doppler sensor 1 is installed at, for example, one corner of a room, transmits a light-based, electromagnetic-based, or acoustic-based transmission wave toward the interior of the room, which is a detection area, and receives a reflection wave (reception wave) reflected by a moving object (reflective object), such as a person or an animal, in the room. Although FIG. 1 shows an example in which one person is present in the room, there may be a plurality of people or may be an animal other than humans.

The Doppler sensor 1 generates a Doppler signal based on the transmission wave and the reception wave and outputs the Doppler signal to the characteristic-value calculating device 2. Then, the characteristic-value calculating device 2 extracts, from the Doppler signal, a characteristic value for more accurately recognizing the state of the moving object in the room and outputs the characteristic value to the recognizing device 3.

The characteristic-value calculating device 2 according to the embodiment of the present invention extracts higher-order local autocorrelation (HLAC) as such a characteristic value. Higher-order local autocorrelation is a characteristic value that expresses a correlation quantity of a single signal or a plurality of signals in local time. In the embodiment of the present invention, higher-order local autocorrelation is applied to a Doppler signal so that a change in phase difference and the periodicity of movement (human body movement or biological signal) within a target area can be extracted.

Based on the characteristic value extracted by the characteristic-value calculating device 2, the recognizing device 3 recognizes whether the room is in an unmanned state or a manned state, and if the room is in a manned state, the recognizing device 3 recognizes whether the room is in an activity state or a rest state. The term “unmanned” refers to a state where there is no one present within the target area. The term “rest” refers to a state where there is a person present within the target area but only breathing without actively moving (e.g., a state where the person is sitting in a chair or on the floor, is standing, or is lying down). The term “activity” refers to a state where there is a person present within the target area and actively moving, such as moving hands and feet (e.g., positionally moving or stamping feet).

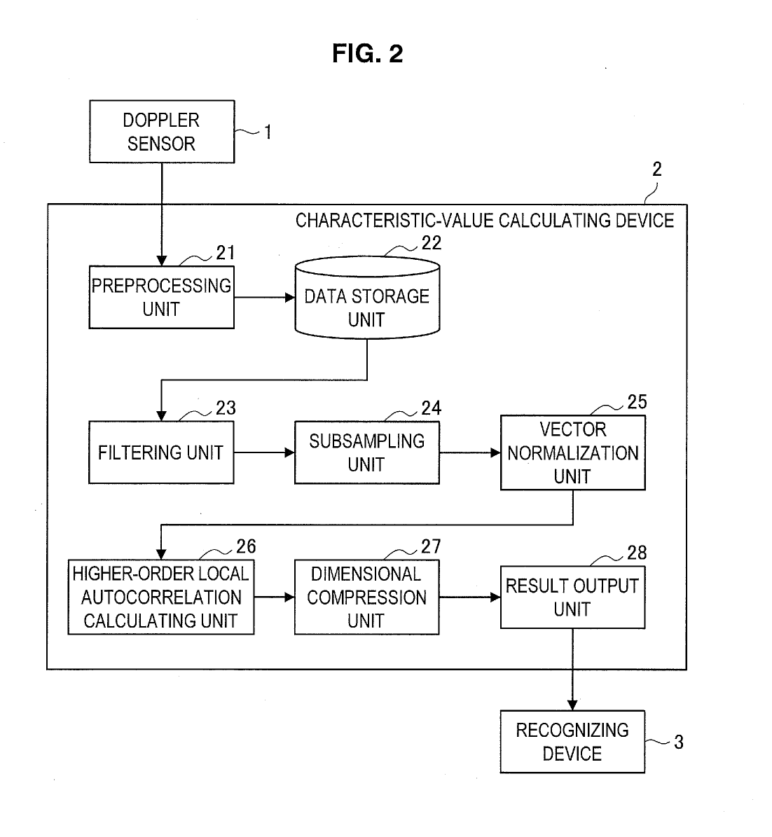

The overview of the state recognizing system has been described above. The state recognizing system according to the embodiment of the present invention will be described in detail below with reference to FIG. 2 to FIG. 6.

### 2. Embodiment of Present Invention

### [2.1. Configuration]

FIG. 2 is a block diagram illustrating the configuration of the state recognizing system according to the embodiment of the present invention. As shown in FIG. 2, the state recognizing system has the Doppler sensor 1,the characteristic-value calculating device 2, and the recognizing device 3.

### (Doppler Sensor 1)

The Doppler sensor 1 has a function of an observing unit that observes a Doppler signal indicating movement of a moving object within a target observation space (detection area). The Doppler sensor 1 has a configuration for emitting an output signal from a local oscillator via a transmission antenna and receiving a reflection wave from a target object via a reception antenna. When the Doppler sensor 1 receives a reflection wave from a target object via the reception antenna, the Doppler sensor 1 divides the reception signal into two signals by using a distributor and delays one of the signals by 90 degrees by using a phase shifter. Since the reflection wave from the moving object is frequency-modulated due to a Doppler effect, a phase difference occurs in the signals.

In this embodiment, the two waves with phases different from each other by 90 degrees, which are obtained by the Doppler sensor 1,will be defined as V_{I}(t) and V_{Q}(t) as shown in Equation 1 below. The subscripts I and Q denote in-phase and quadrature, respectively.

$$\begin{matrix}[ Math . 1 ] & \\ {V}_{I} ( t ) = {A}_{I} sin ( \frac{4 π R ( t )}{λ} + {φ}_{0} ) + {O}_{I} + {w}_{I} \\ {V}_{Q} ( t ) = {A}_{Q} sin ( \frac{4 π R ( t )}{λ} + {φ}_{0} + \frac{π}{2} ) + {O}_{Q} + {w}_{Q} & ( Equation 1 )\end{matrix}$$

In Equation 1, A denotes the amplitude of each signal, λ, denotes the wavelength, R(t) denotes the distance between the Doppler sensor 1 and the target object at a time point t, φ_{O} denotes the initial phase, O denotes direct-current offset, and w denotes a noise component. A method of how Equation 1 is derived is disclosed in “Droitcour, A. D. et al. “Range correlation and I/Q performance benefits in single-chip silicon Doppler radars for noncontact cardiopulmonary monitoring” Microwave Theory and Techniques, IEEE Transactions, Vol. 52, No. 3, pp. 838-848, March 2004″.

The Doppler sensor 1 outputs the signals V_{I}(t) and V_{Q}(t) to the characteristic-value calculating device 2. The signals V_{I}(t) and V_{Q}(t) may also be referred to as Doppler signals hereinafter.

### (Characteristic-Value Calculating Device 2)

The characteristic-value calculating device 2 extracts a characteristic value indicating the state of the target object within the detection area from the Doppler signals output from the Doppler sensor 1. The characteristic-value calculating device 2 functions as a preprocessing unit 21, a data storage unit 22, a filtering unit 23, a subsampling unit 24, a vector normalization unit 25, a higher-order local autocorrelation calculating unit 26, a dimensional compression unit 27, and a result output unit 28.

### Preprocessing Unit 21

The preprocessing unit 21 has a function of an acquiring unit that acquires the Doppler signals from the Doppler sensor 1 and a function for performing predetermined signal processing (preprocessing) on the Doppler signals. The preprocessing executed by the preprocessing unit 21 may include, for example, conversion to digital signals by sampling the signal intensity, offset adjustment of the signals, and removal of direct-current components by applying a high-pass filter. In accordance with the offset adjustment performed by the preprocessing unit 21, the characteristic-value calculating device 2 can also deal with a case where different types of Doppler sensors 1 are connected.

The Doppler sensor 1 and the preprocessing unit 21 are realized as separate hardware units in the configuration shown in FIG. 2 but may also be realized as a single hardware unit. Specifically, the preprocessing unit 21 may be included in the HMD1, or the characteristic-value calculating device 2 may be included in the Doppler sensor 1. Furthermore, the preprocessing unit 21 may perform subsampling on the acquired Doppler signals so as to remove noise components superposed due to supply voltage as well as redundant high-frequency regions, thereby reducing the throughput in subsequent blocks.

The preprocessing unit 21 outputs the preprocessed Doppler signals to the data storage unit 22.

### Data Storage Unit 22

The data storage unit 22 has a function of storing the Doppler signals output from the preprocessing unit 21. The data storage unit 22 is realized by, for example, a hard disc drive (ADD), a solid-state memory such as a flash memory, a memory card containing a solid-state memory, an optical disk, a magneto-optical disk, or a hologram memory.

The data storage unit 22 outputs the stored Doppler signals to the filtering unit 23.

In the present invention, operation may be performed in real-time or in non-real-time. Therefore, the data storage unit 22 may output the Doppler signals output from the preprocessing unit 21 to the filtering unit 23 in real-time, or may output the stored Doppler signals in non-real-time. Furthermore, the preprocessing unit 21 and the data storage unit 22 may be separated from the characteristic-value calculating device 2 and be integrated with the Doppler sensor 1. In this case, the data storage unit 22 stores a signal observed by the Doppler sensor 1 and preprocessed by the preprocessing unit 21, and the subsequent processing is performed by, for example, a separate general-purpose personal computer, so that the device that performs sensing can be reduced in size.

### Filtering Unit 23

The filtering unit 23 has a function of an extracting unit that extracts a time-series signal constituted of a predetermined frequency component from the Doppler signals preprocessed by the preprocessing unit 21. Specifically, the filtering unit 23 performs frequency-filtering on the Doppler signals output from the data storage unit 22.

Although the sampling rate of a Doppler sensor is normally several kHz, a frequency component of human movement is about 0.1 Hz to several tens of Hz. Therefore, the filtering unit 23 sets a cutoff frequency in accordance with a frequency band to which noteworthy components, such as respiration, heartbeat, and body movement, may belong, and applies a band-pass filter or a low-pass filter so as to extract only a signal component deriving from human movement.

The filtering method in the filtering unit 23 is not limited to a specific method. The method used in the filtering unit 23 may be selected from among a method that employs conversion to a frequency domain based on Fourier transform, an infinite impulse response (AIR) filter, and a finite impulse response (FIR) filter, so long as a digital signal can be filtered.

The filtering unit 23 outputs the extracted signal to the subsampling unit 24.

### Subsampling Unit 24

The subsampling unit 24 has a function of a selecting unit that selects a signal value at predetermined intervals from the signal extracted by the filtering unit 23. More specifically, the subsampling unit 24 selects (sub samples) a signal to be used by the higher-order local autocorrelation calculating unit 26, to be described later, from a signal sequence output from the filtering unit 23.

Higher-order local autocorrelation to be calculated by the higher-order local autocorrelation calculating unit 26, to be described later, is defined in Equation 2 below.

$$\begin{matrix}[ Math . 2 ] & \\ {r}_{f} ( {τ}_{1} , … , {τ}_{N} ) = {\sum }_{t} f ( t ) f ( t + {τ}_{1} ) … f ( t + {τ}_{N} ) & ( Equation 2 )\end{matrix}$$

In above Equation 2, τ denotes a correlation width. The subsampling unit 24 selects a plurality of signals to be used when calculating the higher-order local autocorrelation shown in above Equation 2 at intervals of the correlation width τ. In this embodiment, the subsampling unit 24 selects six points, namely, V_{I}(t), V_{I}t+τ), V_{I}(t+2τ), V_{Q}(t), V_{Q}(t+τ), and V_{Q}(t+2τ), which are to be used for calculating second-order higher-order local autocorrelation of the signals V_{I}(t) and V_{Q}(t). As a selection method, for example, in the case of the signal V_{I}(t+τ), the subsampling unit 24 may use a point preceding V_{I}(t) by the correlation width τ, or may use a smoothed signal by taking an average of points before and after the signal by τ/2.

The subsampling unit 24 outputs the signals of the six selected points to the vector normalization unit 25.

### Vector Normalization Unit 25

The vector normalization unit 25 has a function of performing vector normalization on the signals selected by the subsampling unit 24. Specifically, the vector normalization unit 25 performs vector normalization defined in Equation 3 below and unifies the magnitude of the vectors at the six points to 1.

$$\begin{matrix}[ Math . 3 ] & \\ {V}_{I}^{′} ( t ) = \frac{{V}_{I} ( t )}{L} , \\ {V}_{I}^{′} ( t + τ ) = \frac{{V}_{I} ( t + τ )}{L} , \\ {V}_{I}^{′} ( t + 2 τ ) = \frac{{V}_{I} ( t + 2 τ )}{L} , \\ {V}_{Q}^{′} ( t ) = \frac{{V}_{Q} ( t )}{L} , \\ {V}_{Q}^{′} ( t + τ ) = \frac{{V}_{Q} ( t + τ )}{L} , \\ {V}_{Q}^{′} ( t + 2 τ ) = \frac{{V}_{Q} ( t + 2 τ )}{L} \\ where \\ L = \sqrt{\begin{matrix}{{V}_{I} ( t )}^{2} + {{V}_{I} ( t + τ )}^{2} + {{V}_{I} ( t + 2 τ )}^{2} + {{V}_{Q} ( t )}^{2} + \\ {{V}_{Q} ( t + τ )}^{2} + {{V}_{Q} ( t + 2 τ )}^{2}\end{matrix}} & ( Equation 3 )\end{matrix}$$

In accordance with the vector normalization shown in above Equation 3, the vector normalization unit 25 can alleviate a difference in signal intensity based on a difference in distance between the Doppler sensor 1 and a person subjected to detection.

The vector normalization unit 25 outputs the signals V_{I}′(t), V_{I}′(t+τ), V_{I}′(t+2τ), V_{Q}′(t), V_{Q}′(t+τ), and V_{Q}′(t+2τ) of the six vector-normalized points to the higher-order local autocorrelation calculating unit 26.

### Higher-Order Local Autocorrelation Calculating Unit 26

The higher-order local autocorrelation calculating unit 26 has a function of a calculating unit that calculates higher-order local autocorrelation based on the signals output from the vector normalization unit 25. More specifically, based on the signals of the six points output from the vector normalization unit 25, the higher-order local autocorrelation calculating unit 26 calculates a total of 49 patterns of higher-order local autocorrelation defined in Equation 4 below.

### [Math. 4]

H₁(t)=V′_{I}(t), H₂(t)=V′_{I}(t)·V′_{I}(t), H₃(t)=V′_{I}(t)·V′_{I}(t+τ), H₄(t)=V′_{I}(t)·V′_{I}(t+2τ),

H₅(t)=V′_{I}(t)·V′_{I}(t)·V′_{I}(t), H₆(t)=V′_{I}(t)·V′_{I}(t)·V′_{I}(t+τ), H₇(t)=V′_{I}(t)·V′_{I}(t)·V′_{I}(t+2τ),

H₈(t)=V′_{I}(t)·V′_{I}(t+τ)·V′_{I}(t+τ), H₉(t)=V′_{I}(t)·V′_{I}(t+2τ)·V′_{I}(t+2τ),

H₁₀(t)=V′_{I}(t)·V′_{I}(t+τ)·V+_{I}(t+2τ),

H₁₁(t)=V′_{Q}(t), H₁₂(t)=V′_{Q}(t)·V′_{Q}(t), H₁₃(t)=V′_{Q}(t)·V′_{Q}(t+τ), H₁₄(t)=V′_{Q}(t)V′_{Q}(t+2τ),

H₁₅(t)=V′_{Q}(t)·V′_{Q}(t)·V′_{Q}(t), H₁₆(t)=V′_{Q}(t)·V′_{Q}(t)·V′_{Q}(t+τ), H₁₇(t)=V′_{Q}(t)·V′_{Q}(t)·V′_{Q}(t+2τ),

H₁₈(t)=V′_{Q}(t)·V′_{Q}(t+τ)·V′_{Q}(t+τ), H₁₉(t)=V′_{Q}(t)·V′_{Q}(t+2τ)·V′_{Q}(t+2τ),

H₂₀(t)=V′_{Q}(t)·V′_{Q}(t+τ)·V′_{Q}(t+2τ),

H₂₁(t)=V′_{I}(t)·V′_{Q}(t), H₂₂(t)=V′_{I}(t)·V′_{Q}(t+τ), H₂₃(t)=V′_{I}(t)·V′_{Q}(t+2τ),

H₂₄(t)=V′_{I}(t+τ)·V′_{Q}(t), H₂₅(t)=V′_{I}(t+τ)·V′_{Q}(t),

H₂₆(t)=V′_{I}(t)·V′_{Q}(t), H₂₇(t)=V′_{I}(t)·V′_{I}(t)·V′_{Q}(t+τ), H₂₈(t)=V′_{I}(t)·V′_{Q}(t+2τ),

H₂₉(t)=V′_{I}(t+τ)·V′_{I}(t+τ)·V′_{Q}(t), H₃₀ (t)=V′_{I}(t+2τ)·V′_{I}(t+2τ)·V′_{Q}(t),

H₃₁(t)=V′_{I}(t)·V′_{I}(t+τ)·V′_{Q}(t), H₃₂(t)=V′_{I}(t)·V′_{I}(t+2τ)·V′_{Q}(t),

H₃₃(t)=V′_{I}(t)·V′_{I}(t+τ)·V′_{Q}(t+τ), H₃₄(t)=V′_{I}(t)·V′_{I}(t+τ)·V′_{Q}(t+2τ),

H₃₅(t)=V′_{I}(t)·V′_{I}(t+2τ)·V′_{Q}(t+τ), H₃₆(t)=V′_{I}(t)·V′_{I}(t+2τ)·V′_{Q}(t+2τ),

H₃₇(t)=V′_{I}(t+τ)·V′_{I}(t+2τ)·V′_{Q}(t),

H₃₈(t)=V′_{Q}(t)·V′_{Q}(t)·V′_{I}(t), H₃₉(t)=V′_{Q}(t)·V′_{Q}(t)·V′_{I}(t+τ), H₄₀(t)=V′_{Q}(t)·V′_{Q}(t)·V′_{I}(t+2τ),

H₄₁(t)=V′_{Q}(t+τ)·V′_{Q}(t+τ)·V′_{I}(t), H₄₂(t)=V′_{Q}(t+2τ)·V′_{Q}(t+2τ)·V_{I}(t),

H₄₃(t)=V′_{Q}(t)·V′_{Q}(t+τ)·V′_{I}(t), H₄₄(t)=V′_{Q}(t)·V′_{Q}(t+2τ)·V′_{I}(t),

H₄₅(t)=V′_{Q}(t)·V′_{Q}(t+τ)·V′_{I}(t+τ), H₄₆(t)=V′_{Q}(t)·V′_{Q}(t+τ)·V′_{I}(t+2τ),

H₄₇(t)=V′_{Q}(t)·V′_{Q}(t+2τ)·V′_{I}(t+τ), H₄₈(t)=V′_{Q}(t)·V′_{Q}(t+2τ)·V′_{I}(t+2τ),

H₄₉(t)=V′_{Q}(t+τ)·V′_{Q}(t+2τ)·V′_{I}(t),

The higher-order local autocorrelation calculating unit 26 outputs the calculated 49 patterns of higher-order local autocorrelation to the dimensional compression unit 27.

### Dimensional Compression Unit 27

The dimensional compression unit 27 has a function of performing dimensional compression on the higher-order local autocorrelation output from the higher-order local autocorrelation calculating unit 26. For example, the dimensional compression unit 27 performs dimensional compression by using a dimensional compression method such as a principal component analysis. Therefore, the dimensional compression unit 27 can specify an efficient characteristic value for state recognition by the recognizing device 3, to be described later.

The dimensional compression unit 27 outputs the dimensional compression result to the result output unit 28.

### Result Output Unit 28

The result output unit 28 outputs the result output from the dimensional compression unit 27 to the recognizing device 3 as a characteristic value indicating the state within the detection area. Alternatively, the result output unit 28 may directly output the 49 patterns of higher-order local autocorrelation calculated by the higher-order local autocorrelation calculating unit 26 as a characteristic value indicating the state within the detection area.

### (Recognizing Device 3)

The recognizing device 3 has a function of a recognizing unit that recognizes the state of the detection area of the Doppler sensor 1 based on the characteristic value (higher-order local autocorrelation) output from the result output unit 28. The recognizing device 3 recognizes at least one of unmanned, rest, and activity states as the state of the space.

Specifically, the recognizing device 3 first stores the characteristic value output from the result output unit 28 while clustering the characteristic value together with annotations such as the unmanned, rest, and activity states. Then, the recognizing device 3 calculates the degree of similarity between the characteristic value output from the result output unit 28 and an aggregate of clustered state vectors and recognizes similar annotations as the state of the detection area. For calculating the degree of similarity, the recognizing device 3 may use, for example, a recognizer that is based on a support vector machine or a hidden Marks model.

The recognizing device 3 may give the rest and activity states a generic name of “manned” state so as to simply recognize one of two states, that is, an unmanned state and a manned state. Furthermore, the recognizing device 3 may be integrated with the characteristic-value calculating device 2.

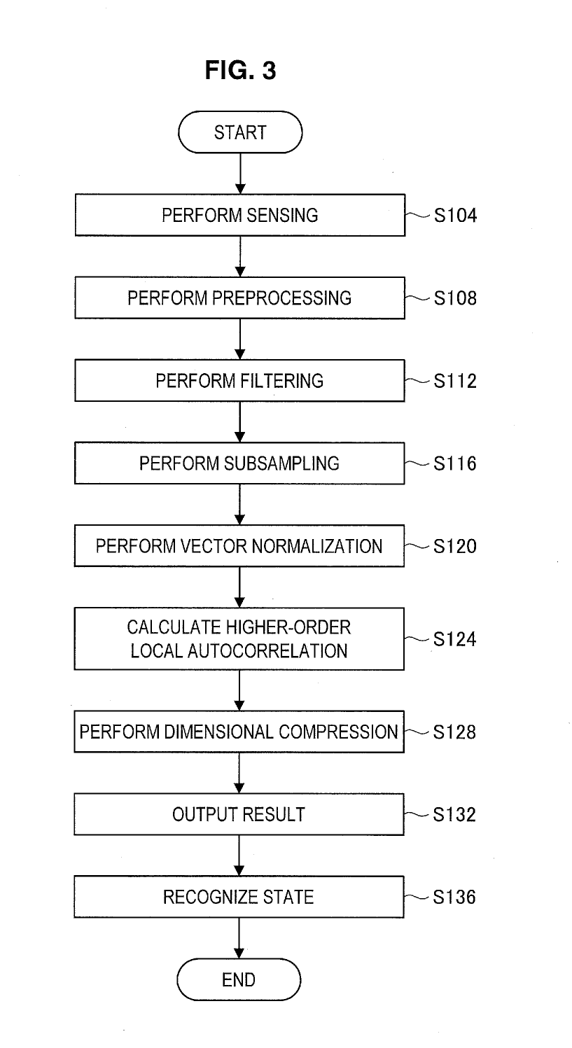

The configuration of the state system according to this embodiment has been described above. Next, an operation of the state system according to this embodiment will be described with reference to FIG. 3.

### [2.2. Operation]

FIG. 3 is a flowchart illustrating the operation of the state recognizing system according to the embodiment of the present invention. As shown in FIG. 3, in step S104, the Doppler sensor 1 first performs sensing in the detection area. Specifically, the Doppler sensor 1 emits an output signal from the local oscillator via the transmission antenna, receives a reflection wave from a target object via the reception antenna, and obtains two waves of Doppler signals V_{I}(t) and V_{Q}(t) shown in Equation 1 mentioned above based on the reflection wave and the reception wave.

Then, in step S108, the preprocessing unit 21 performs preprocessing on the two waves of Doppler signals V_{I}(t) and V_{Q}(t) output from the Doppler sensor 1. The preprocessing executed by the preprocessing unit 21 includes, for example, conversion to digital signals by sampling the signal intensity, offset adjustment of the signals, and removal of direct-current components by applying a high-pass filter.

The Doppler signals preprocessed by the preprocessing unit 21 are stored by the data storage unit 22. The data storage unit 22 may output the Doppler signals output from the preprocessing unit 21 to the filtering unit 23 in real-time, or may output the stored Doppler signals in non-real-time.

Subsequently, in step S112, the filtering unit 23 performs filtering for extracting a frequency component deriving from human movement from the Doppler signals output from the data storage unit 22. For example, as a frequency band to which noteworthy components, such as respiration, heartbeat, and body movement, may belong, the filtering unit 23 applies a band-pass filter or a low-pass filter so as to extract only a frequency component ranging between 0.1 Hz and several tens of Hz.

Then, in step S116, the subsampling unit 24 performs subsampling from a signal sequence filtered by the filtering unit 23. Specifically, the subsampling unit 24 selects six points, namely, V_{I}(t), V_{I}(t+τ), V_{I}(t+2τ), V_{Q}(t), V_{Q}(t+τ), and V_{Q}(t+2τ), shown in Equation 3 mentioned above.

Subsequently, in step S 120, the vector normalization unit 25 performs vector normalization on the signals output from the subsampling unit 24. Specifically, the vector normalization unit 25 performs vector normalization shown in Equation 3 mentioned above on the signals of the six points so as to alleviate a difference in signal intensity based on a difference in distance between the Doppler sensor 1 and a person subjected to detection.

Then, in step S114, the higher-order local autocorrelation calculating unit 26 calculates higher-order local autocorrelation based on the signals output from the vector normalization unit 25. Specifically, based on the signal values of the six points, the higher-order local autocorrelation calculating unit 26 calculates 49 patterns of higher-order local autocorrelation shown in Equation 4 mentioned above.

Subsequently, in step S108, the dimensional compression unit 27 performs dimensional compression on the higher-order local autocorrelation output from the higher-order local autocorrelation calculating unit 26. Specifically, the dimensional compression unit 27 specifies an efficient characteristic value for state recognition from the 49 patterns of higher-order local autocorrelation output from the higher-order local autocorrelation calculating unit 26 by, for example, a principal component analysis.

Then, in step S102, the result output unit 28 outputs the characteristic value output from the dimensional compression unit 27. In this case, the result output unit 28 may output the result of the dimensional compression process by the dimensional compression unit 27 in the form of, for example, data, text, audio, or an image.

Subsequently, in step S106, the recognizing device 3 recognizes the state of the detection area of the Doppler sensor 1 based on the characteristic value output from the result output unit 28. Specifically, the recognizing device 3 recognizes whether the state of the space is any one of unmanned, rest, and activity states by applying a recognizer that is based on a support vector machine or a hidden Marks model.

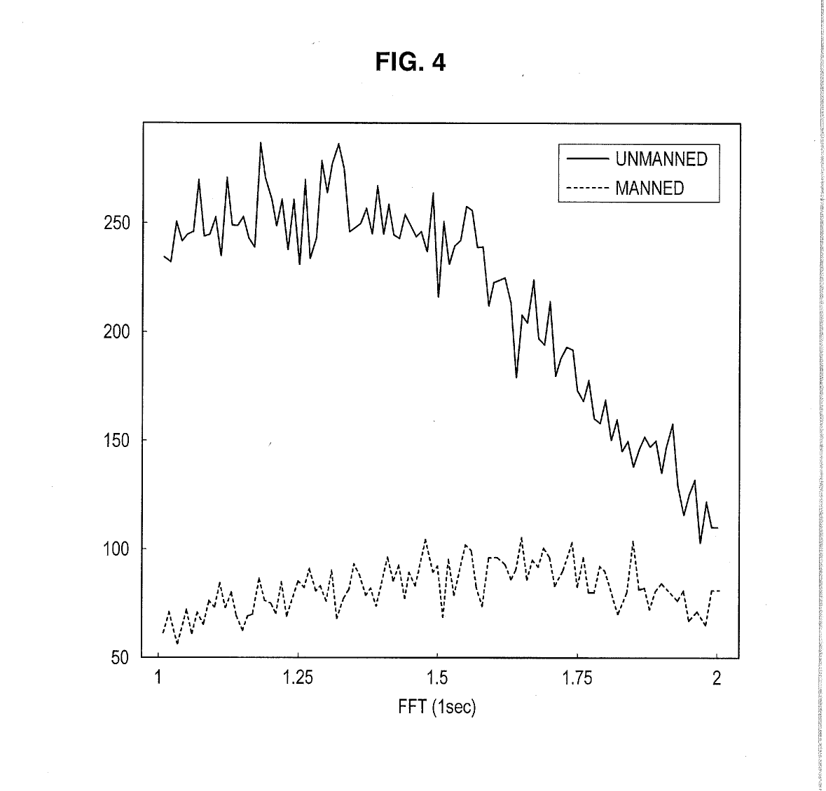

The operation of the state system according to this embodiment has been described above. Next, advantages exhibited by the state system according to this embodiment will be described with reference to FIG. 4 to FIG. 6.

### [2.3. Advantages]

By using higher-order local autocorrelation, the characteristic-value calculating device 2 according to this embodiment can extract a characteristic value, such as a change in phase difference and the periodicity of movement (human body movement or a biological signal) within a target area, different from signal amplitude or frequency. Therefore, the recognizing device 3 can distinguish a human from other disturbance even in a case where signal intensity similar to a Doppler signal obtained from human biological information is obtained, such as when there is a small animal, or in a case where there is an electrical household device that operates in a cycle similar to the human respiration cycle. Thus, the recognizing device 3 can more accurately detect the state within the detection area based on the characteristic value calculated by the characteristic-value calculating device 2.

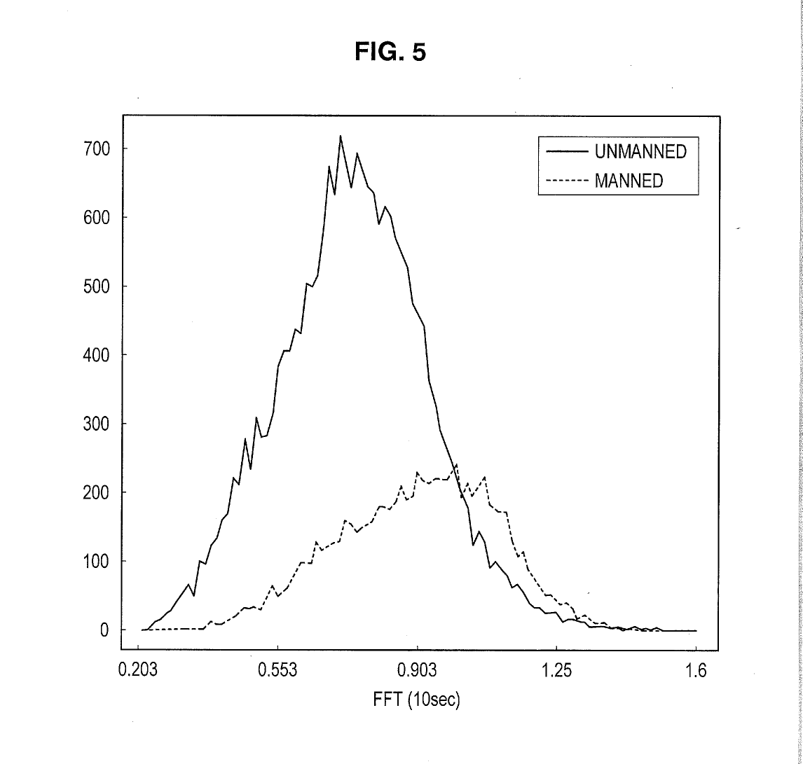

The advantages exhibited by the state system according to this embodiment will be described below in comparison with a technique for extracting a characteristic value by fast Fourier transform (FFT), which is one of techniques in the related art, as a comparative example. In the comparative example, a Doppler signal in a predetermined interval is converted into a frequency domain by FFT, and an average frequency of the same frequency domain as the characteristic-value calculating device 2 is calculated as a characteristic value. FIG. 4 and FIG. 5 illustrate the distribution of manned and unmanned states of average frequencies calculated in the comparative example.

FIG. 4 is a diagram illustrating frequency distribution of average frequencies by FFT in a one-second interval. FIG. 5 is a diagram illustrating frequency distribution of average frequencies by FFT in a ten-second interval. As shown in FIG. 4, with FFT in the one-second interval, it is difficult to determine differences in characteristic values based on manned and unmanned states. On the other hand, as shown in FIG. 5, with FFT in the ten-second interval, it is clear that the manned-state characteristic values are distributed as larger values than the unmanned-state characteristic values. In other words, the characteristic values according to FFT in the ten-second interval are considered to be useful for determining manned and unmanned states. However, it can also be said that characteristic values according to FFT in the examples shown in FIG. 4 and FIG. 5 become useful for determining manned and unmanned states only after there is data from a long interval of about ten seconds.

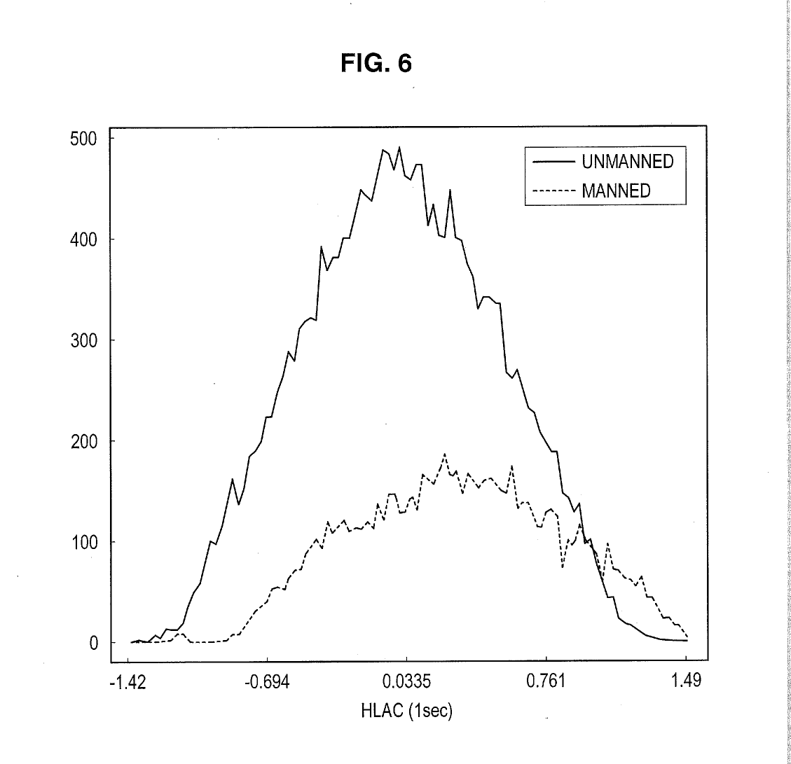

In contrast, the higher-order local autocorrelation according to the embodiment of the present invention is useful for determining manned and unmanned states even with data from a short interval of about one second. FIG. 6 illustrates the distribution of manned and unmanned states with respect to a first principal component of higher-order local autocorrelation calculated by the characteristic-value calculating device 2 relative to a Doppler signal in a one-second interval when the correlation width t is set to 0.5 seconds (250 samples if the sampling frequency is 500 Hz).

FIG. 6 is a diagram illustrating frequency distribution of the first principal component of higher-order local autocorrelation in a one-second interval in accordance with the embodiment of the present invention. As shown in FIG. 6, it is clear that the manned-state characteristic values are distributed as larger values than the unmanned-state characteristic values even in the one-second interval. Accordingly, the characteristic-value calculating device 2 according to the embodiment of the present invention can calculate a characteristic value useful for determining manned and unmanned states even with data from a short interval, with which it is difficult to calculate a useful characteristic value in the comparative example.

Furthermore, for example, in a case where the sampling frequency is set to 500 Hz, the number of samples subjected to calculation by FFT in the one-second interval is 500 in the comparative example. In contrast, in the characteristic-value calculating device 2 according to the embodiment of the present invention, six points, namely, V_{I}(t), V_{I}(t+τ), V_{I}(t+2τ), V_{Q}(t), V_{Q}(t+τ), and V_{Q}(t+2τ), are subjected to calculation even in the one-second interval.

In other words, the characteristic-value calculating device 2 according to the embodiment of the present invention can significantly reduce the number of samples subjected to calculation, as compared with the comparative example, and can consequently reduce the calculation amount for calculating a characteristic value.

The advantages exhibited by the state system according to this embodiment have been described above.

<3. Conclusion>As described above, the characteristic-value calculating device 2 according to this embodiment can extract a characteristic value for more accurately recognizing the state of a moving object, such as a human, from a Doppler signal. Moreover, the characteristic-value calculating device 2 can calculate a useful characteristic value with short-interval data, as compared with the technique in the related art. Furthermore, the characteristic-value calculating device 2 can reduce the calculation amount for calculating a characteristic value, as compared with the technique in the related art.

Heretofore, preferred embodiments of the present invention have been described in detail with reference to the appended drawings, but the present invention is not limited thereto. It should be understood by those skilled in the art that various changes and alterations may be made without departing from the spirit and scope of the appended claims.

For example, although the state of the detection area with a human as a target object is recognized in the above embodiment, the present invention is not limited to this example. For example, the characteristic-value calculating device 2 may set a component to be filtered by the filtering unit 23 as a frequency band arising from biological information, such as respiration or heartbeat of an animal other than humans, so as to set an arbitrary moving object other than humans as a target object.

Furthermore, a computer program for causing hardware units, such as a CPU, a ROM, and a RAM, contained in an information processing device to exhibit functions similar to the components in the above-described state recognizing system can also be created. Moreover, a recording medium having such a computer program stored therein is also provided.

### REFERENCE SIGNS LIST

1 Doppler sensor
2 characteristic-value calculating device
21 preprocessing unit
22 data storage unit
23 filtering unit
24 subsampling unit
25 vector normalization unit
26 higher-order local autocorrelation calculating unit
27 dimensional compression unit
28 result output unit
3 recognizing device

1. A characteristic-value calculating device comprising:
an acquiring unit that acquires a Doppler signal;
an extracting unit that extracts a time-series signal constituted of a predetermined frequency component from the Doppler signal acquired by the acquiring unit;
a selecting unit that selects a signal value at a predetermined interval from the time-series signal extracted by the extracting unit; and
a calculating unit that calculates higher-order local autocorrelation based on the signal value selected by the selecting unit.

2. The characteristic-value calculating device according to claim 1, further comprising:
a vector normalization unit that performs vector normalization on the signal value selected by the selecting unit, wherein the calculating unit calculates the higher-order local autocorrelation based on the signal value normalized by the vector normalization unit.

3. The characteristic-value calculating device according to claim 1, further comprising:
a dimensional compression unit that performs dimensional compression on the higher-order local autocorrelation calculated by the calculating unit.

4. The characteristic-value calculating device according to claim 1, further comprising:
a preprocessing unit that performs predetermined signal processing on the Doppler signal acquired by the acquiring unit,
wherein the extracting unit extracts the time-series signal from the Doppler signal signal-processed by the preprocessing unit.

5. The characteristic-value calculating device according to claim 4,
wherein the preprocessing unit performs offset adjustment on the Doppler signal acquired by the acquiring unit.

6. The characteristic-value calculating device according to claim 1,
wherein the extracting unit extracts the time-series signal constituted of a frequency component arising from human movement from the Doppler signal acquired by the acquiring unit.

7. The characteristic-value calculating device according to claim 1, further comprising:
a recognizing unit that recognizes a state of a space subjected to observation of the Doppler signal acquired by the acquiring unit based on the higher-order local autocorrelation calculated by the calculating unit.

8. The characteristic-value calculating device according to claim 7,
wherein the recognizing unit recognizes at least one of unmanned, rest, and activity states as the state of the space.

9. The characteristic-value calculating device according to claim 1, further comprising:
an observing unit that observes the Doppler signal and outputs the Doppler signal to the acquiring unit.

10. A characteristic-value calculating method comprising:
a step for acquiring a Doppler signal;
a step for extracting a time-series signal constituted of a predetermined frequency component from the acquired Doppler signal;
a step for selecting a signal value at a predetermined interval from the extracted time-series signal; and
a step for calculating higher-order local autocorrelation based on the selected signal value.

11. A recording medium having a program stored therein, the program causing a computer to execute:
a step for acquiring a Doppler signal;
a step for extracting a time-series signal constituted of a predetermined frequency component from the acquired Doppler signal;
a step for selecting a signal value at a predetermined interval from the extracted time-series signal; and
a step for calculating higher-order local autocorrelation based on the selected signal value.
